(19) United States
(12) Patent Application Publication
(10) Pub. No.: US 2005/0260246 A1
(43) Pub. Date: Nov. 24, 2005

(54) Bioactive agent release coating

(76) Inventors: Stephen J. Chudzik, St. Paul, MN (US); Aron B. Anderson, Minnetonka, MN (US); Ralph A. Chappa, Prior Lake, MN (US); Timothy M. Kloke, Chaska, MN (US); Laurie R. Lawin, New Brighton, MN (US); Klaus R. Wormuth, Minneapolis, MN (US)

Correspondence Address: SURMODICS, INC., 9924 WEST 74TH STREET, EDEN PRAIRIE, MN, 55344, US

(21) Appl. No.: 11/123,835

(22) Filed: May 6, 2005

Related U.S. Application Data
(63) Continuation-in-part of application No. 10/268,163, filed on Oct. 10, 2002.
Application No. 10/268,163 is a continuation of application No. 09/989,033, filed on Nov. 21, 2001, now Pat. No. 6,890,583.
Application No. 09/989,033 is a division of application No. 09/693,771, filed on Oct. 20, 2000, now Pat. No. 6,344,035.
Application No. 09/693,771 is a division of application No. 09/292,510, filed on Apr. 15, 1999, now Pat. No. 6,214,901.
(60) Provisional application No. 60/083,135, filed on Apr. 27, 1998.

(51) Int. Cl. A61F 2/02

(52) U.S. Cl. 424/423

(57) ABSTRACT

A coating composition for use in coating implantable medical devices to improve their ability to release bioactive agents in vivo. The coating composition is particularly adapted for use with devices that undergo significant flexion and/or expansion in the course of their delivery and/or use, such as stents and catheters. The composition includes the bioactive agent in combination with a mixture of a first polymer component such as poly(butyl methacrylate) and a second polymer component such as poly(ethylene-co-vinyl acetate).

### CROSS REFERENCE TO RELATED APPLICATIONS

The present application is a continuation-in-part of a U.S. patent application filed Oct. 10, 2002 and assigned Ser. No. 10/268,163, which is a divisional of U.S. patent application filed Nov. 21, 2001 and assigned Ser. No. 09/989,033, which is a divisional of U.S. patent application filed Oct. 20, 2000 and assigned Ser. No. 09/693,771 (now U.S. Pat. No. 6,344,035, issued Feb. 5, 2002), which is a divisional of U.S. patent application filed Apr. 15, 1999 and assigned Ser. No. 09/292,510 (now U.S. Pat. No. 6,214,901, issued Apr. 10, 2001), which is a continuation-in-part of provisional U.S. patent application filed Apr. 27, 1998 and assigned Ser. No. 60/083,135, the entire disclosures of which are incorporated herein by reference.

### TECHNICAL FIELD

In one aspect, the present invention relates to a process of treating implantable medical devices with coating compositions to provide the release of pharmaceutical agents from the surface of the devices under physiological conditions. In another aspect, the invention relates to the coating compositions, per se, and to devices coated with such compositions.

### BACKGROUND OF THE INVENTION

Many surgical interventions require the placement of a medical device into the body. While necessary and beneficial for treating a variety of medical conditions, the placement of metal or polymeric devices in the body gives rise to numerous complications. Some of these complications include: increased risk of infection; initiation of a foreign body response resulting in inflammation and fibrous encapsulation; and initiation of a wound healing response resulting in hyperplasia and restenosis. These and other complications must be dealt with when introducing a metal or polymeric device into the body.

One approach to reducing the potential harmful effects of such an introduction is to attempt to provide a more biocompatible implantable device. While there are several methods available to improve the biocompatibility of implantable devices, one method which has met with limited success is to provide the device with the ability to deliver bioactive compounds to the vicinity of the implant. By so doing, some of the harmful effects associated with the implantation of medical devices can be diminished. Thus, for example, antibiotics can be released from the surface of the device to minimize the possibility of infection, and anti-proliferative drugs can be released to inhibit hyperplasia. Another benefit to the local release of bioactive agents is the avoidance of toxic concentrations of drugs which are sometimes necessary, when given systemically, to achieve therapeutic concentrations at the site where they are needed.

Although the potential benefits expected from the use of medical devices capable of releasing pharmaceutical agents from their surfaces is great, the development of such medical devices has been slow. This development has been hampered by the many challenges that need to be successfully overcome when undertaking said development. Some of these challenges are: 1) the requirement, in some instances, for long term release of bioactive agents; 2) the need for a biocompatible, non-inflammatory device surface; 3) the need for significant durability, particularly with devices that undergo flexion and/or expansion when being implanted or used in the body; 4) concerns regarding processability, to enable the device to be manufactured in an economically viable and reproducible manner; and 5) the requirement that the finished device be sterilizable using conventional methods.

Several implantable medical devices capable of delivering medicinal agents have been described. Several patents are directed to devices utilizing biodegradable or bioresorbable polymers as drug containing and releasing coatings, including Tang et al, U.S. Pat. No. 4,916,193 and MacGregor, U.S. Pat. No. 4,994,071. Other patents are directed to the formation of a drug containing hydrogel on the surface of an implantable medical device, these include Amiden et al, U.S. Pat. No. 5,221,698 and Sahatjian, U.S. Pat. No. 5,304,121. Still other patents describe methods for preparing coated intravascular stents via application of polymer solutions containing dispersed therapeutic material to the stent surface followed by evaporation of the solvent. This method is described in Berg et al, U.S. Pat. No. 5,464,650.

However, there remain significant problems to be overcome in order to provide a therapeutically significant amount of a bioactive compound on the surface of the implantable medical device. This is particularly true when the coating composition must be kept on the device in the course of flexion and/or expansion of the device during implantation or use. It is also desirable to have a facile and easily processable method of controlling the rate of bioactive release from the surface of the device.

Although a variety of hydrophobic polymers have previously been described for use as drug release coatings, Applicant has found that only a small number possess the physical characteristics that would render them useful for implantable medical devices which undergo flexion and/or expansion upon implantation. Many polymers which demonstrate good drug release characteristics, when used alone as drug delivery vehicles, provide coatings that are too brittle to be used on devices which undergo flexion and/or expansion. Other polymers can provoke an inflammatory response when implanted. These or other polymers demonstrate good drug release characteristics for one drug but very poor characteristics for another.

Some polymers show good durability and flexibility characteristics when applied to devices without drug, but lose these favorable characteristics when drug is added. Furthermore, often times the higher the concentration of drugs or the thicker the application of polymer to the device surface, the poorer the physical characteristics of the polymer become. It has been very difficult to identify a polymer which provides the proper physical characteristics in the presence of drugs and one in which the drug delivery rate can be controlled by altering the concentration of the drug in the polymer or the thickness of the polymer layer.

There remains a need, therefore, for an implantable medical device that can undergo flexion and/or expansion upon implantation, and that is also capable of delivering a therapeutically significant amount of a pharmaceutical agent or agents from the surface of the device.

### BRIEF DESCRIPTION OF THE DRAWINGS

In the Drawings:

FIG. 1 provides a plot showing the cumulative release profiles for wires coated with compositions according to the present invention, as described in Example 1.

FIG. 2 is a Darkfield image of a medical device surface coating of the present invention.

FIG. 3 is a Darkfield image of a medical device surface coating showing cloudy areas.

FIG. 4 is a Scanning Electron Microscope image of the coating corresponding to FIG. 2.

FIG. 5 is a Scanning Electron Microscope image of the coating corresponding to FIG. 3.

FIG. 6 is an image of a stent portion coating using features of the present invention.

FIG. 7 is an image of a stent portion coating not employing the present invention.

### SUMMARY OF THE INVENTION

The present invention provides a coating composition and related method for using the composition to coat an implantable medical device with a bioactive agent in a manner that permits the surface to release the bioactive agent over time when implanted in vivo. In a particularly preferred embodiment, the device is one that undergoes flexion and/or expansion in the course of implantation or use in vivo.

The composition comprises a bioactive agent in combination with a plurality of polymers, including a first polymer component and a second polymer component. The polymer components are adapted to be mixed to provide a mixture that exhibits an optimal combination of physical characteristics (e.g., adherence, durability, flexibility) and bioactive release characteristics as compared to the polymers when used alone or in admixture with other polymers previously known. In a preferred embodiment the composition comprises at least one poly(alkyl)(meth)acrylate, as a first polymeric component and poly(ethylene-co-vinyl acetate) (“pEVA”) as a second polymeric component.

The composition and method can be used to control the amount and rate of bioactive agent (e.g., drug) release from one or more surfaces of implantable medical devices. In a preferred embodiment, the method employs a mixture of hydrophobic polymers in combination with one or more bioactive agents, such as a pharmaceutical agent, such that the amount and rate of release of agent(s) from the medical device can be controlled, e.g., by adjusting the relative types and/or concentrations of hydrophobic polymers in the mixture. For a given combination of polymers, for instance, this approach permits the release rate to be adjusted and controlled by simply adjusting the relative concentrations of the polymers in the coating mixture. This obviates the need to control the bioactive release rate by polymer selection, multiple coats, or layering of coats, and thus greatly simplifies the manufacture of bioactive-releasing implantable medical devices.

A preferred coating of this invention includes a mixture of two or more polymers having complementary physical characteristics, and a pharmaceutical agent or agents applied to the surface of an implantable medical device which undergoes flexion and/or expansion upon implantation or use. The applied coating is cured (e.g., solvent evaporated) to provide a tenacious and flexible bioactive-releasing coating on the surface of the medical device. The complementary polymers are selected such that a broad range of relative polymer concentrations can be used without detrimentally affecting the desirable physical characteristics of the polymers. By use of the polymer mixtures of the invention the bioactive release rate from a coated medical device can be manipulated by adjusting the relative concentrations of the polymers. Similarly, a spectrum of pharmaceutical agents can be delivered from the coating without the need to find a new polymer or layering the coating on the device.

### DETAILED DESCRIPTION OF THE INVENTION

The present invention relates to a coating composition and related method for coating an implantable medical device which undergoes flexion and/or expansion upon implantation. The structure and composition of the underlying device can be of any suitable, and medically acceptable, design and can be made of any suitable material that is compatible with the coating itself. The surface of the medical device is provided with a coating containing one or more bioactive agents.

In order to provide a preferred coating, a composition is prepared to include a solvent, a combination of complementary polymers dissolved in the solvent, and the bioactive agent or agents dispersed in the polymer/solvent mixture. The solvent is preferably one in which the polymers form a true solution or a mixture or blend with particles so fine as to function as a solution. The pharmaceutical agent itself may either be soluble in the solvent or form a dispersion throughout the solvent.

The resultant composition can be applied to the device in any suitable fashion, e.g., it can be applied directly to the surface of the medical device, or alternatively, to the surface of a surface-modified medical device, by dipping, spraying, or any conventional technique. The method of applying the coating composition to the device is typically governed by the geometry of the device and other process considerations. The coating is subsequently cured by evaporation of the solvent. The curing process can be performed at room temperature, elevated temperature, or with the assistance of vacuum.

The polymer mixture for use in this invention is preferably biocompatible, e.g., such that it results in no induction of inflammation or irritation when implanted. In addition, the polymer combination must be useful under a broad spectrum of both absolute concentrations and relative concentrations of the polymers. This means that the physical characteristics of the coating, such as tenacity, durability, flexibility and expandability, will typically be adequate over a broad range of polymer concentrations. Furthermore, the ability of the coating to control the release rates of a variety of pharmaceutical agents can preferably be manipulated by varying the absolute and relative concentrations of the polymers.

As previously indicated, the polymers utilized in various embodiments of the present invention are generally complimentary to each other and also complimentary to the one or more bioactive agents present in the various polymer blends.

In various embodiments of the present invention, the coating compositions of the present invention may include a variety of polymers as long as at least two are miscible as defined herein to form a polymer blend. The bioactive agents may be incorporated within the miscible polymer blend such that it is delivered from the blend, or the blend can initially function as a barrier to the environment through which the active agent passes.

Miscible polymer blends are advantageous because they can provide greater versatility and tunability for a greater range of bioactive agents than can conventional systems that include immiscible mixtures or only a single polymer, for example. That is, using two or more polymers, at least two of which are miscible, can generally provide a more versatile bioactive agent delivery system than a delivery system with only one of the polymers. A greater range of types of bioactive agents can typically be used. A greater range of amounts of bioactive agents can typically be incorporated into and delivered from (preferably, predominantly under permeation control) the coatings of the present invention. A greater range of delivery rates for the bioactive agents can typically be provided by the coatings of the present invention. At least in part, this is because of the use of a miscible polymer blend that includes at least two miscible polymers. It should be understood that, although the description herein refers to two polymers, the invention may encompass systems that include more than two polymers, as long as a functionally miscible polymer blend is formed that includes at least two complementary polymers.

A miscible polymer blend of the present invention has a sufficient amount of at least two complementary polymers to form a continuous portion, which helps tune the rate of release of the active agent. Such a continuous portion (i.e., continuous phase) can be identified microscopically or by selective solvent etching. Preferably, the at least two complementary polymers form at least 50 percent by volume of a functionally miscible polymer blend.

A miscible polymer blend can also optionally include a dispersed (i.e., discontinuous) immiscible portion. If both continuous and dispersed portions are present, the bioactive agent can be incorporated within either portion. Preferably, the bioactive agent is loaded into the continuous portion to provide delivery of the bioactive agent predominantly under permeation control. To load the bioactive agent, the solubility parameters of the bioactive agent and the portion of the miscible polymer blend a majority of the bioactive agent is loaded into are matched (typically to within no greater than about 10 J.sup.1/2/cm.sup.3/2, preferably, no greater than about 5 J.sup.1/2/cm.sup.3/2, and more preferably, no greater than about 3 J.sup.1/2/cm.sup.3/2). The continuous phase controls the release of the bioactive agent regardless of where the bioactive agent is loaded.

In one embodiment, a miscible polymer blend, as used herein, encompasses a number of completely miscible blends of two or more polymers as well as partially miscible blends of two or more polymers. A completely miscible polymer blend will ideally have a single glass transition temperature (Tg), preferably one in each phase (typically a hard phase and a soft phase) for segmented polymers, due to mixing at the molecular level over the entire concentration range. Partially miscible polymer blends may have multiple Tg's, which can be in one or both of the hard phase and the soft phase for segmented polymers, because mixing at the molecular level is limited to only parts of the entire concentration range. These partially miscible blends are included within the scope of the term “miscible polymer blend” as long as the absolute value of the difference in at least one Tg (Tg_{polymer 1}−Tg_{polymer 2}) for each of at least two polymers within the blend is reduced by the act of blending. Tg's can be determined by measuring the mechanical properties, thermal properties, electric properties, etc. as a function of temperature.

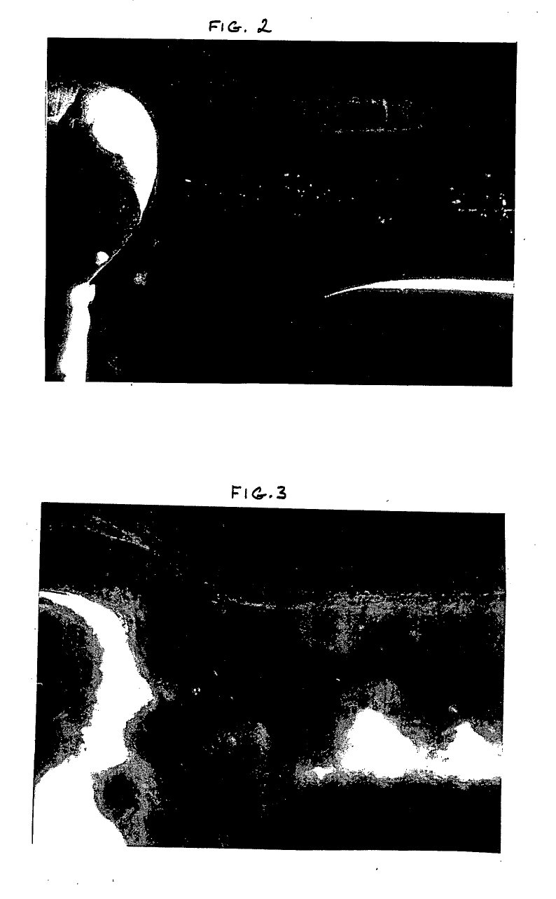

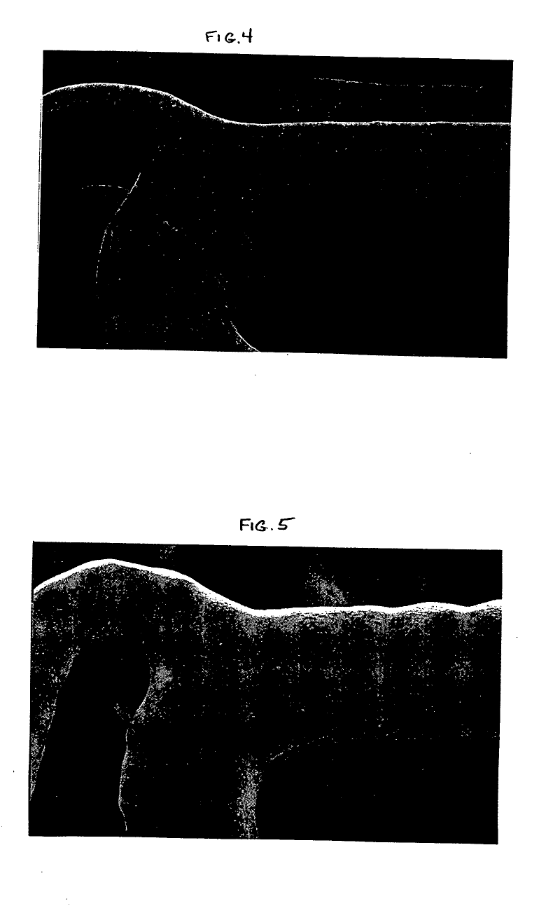

A miscible polymer blend can also be determined based on its optical properties. A completely miscible blend forms a stable and homogeneous domain that is transparent, whereas an immiscible blend forms a heterogeneous domain that scatters light and visually appears turbid unless the components have identical refractive indices. The turbidity may also indicate the presence of a different phase or the location of zones of bioactive agent which has not solubilized. FIG. 2 shows a Darkfield image of a medical device with a coating of the invention having no cloudiness in the coating. FIG. 3 shows a medical device with areas of patchy cloudiness. FIG. 4 is an SEM image of the coating corresponding to that shown in FIG. 2 with a very smooth coating texture and no bumps or surface roughness. FIG. 5 is an SEM image of the coating corresponding to that shown in FIG. 3. FIG. 5 shows bumps and roughness in areas correlating to the areas of cloudiness in FIG. 3. It has been discovered that due to the complementary nature of the numerous polymer blends possible with the teachings of this invention, it is often difficult to analyze the coating quality using standard analysis of Raman microscopy. That analysis typically uses a peak integration and center of mass methodology. However, since the Raman spectra of certain complementary blends overlap strongly, unambiguous separation of these complementary polymers is difficult. This is further complicated by the subtle differences between amorphous or crystalline forms of a bioactive agent. Accordingly, recognizing that this technique is inadequate for certain compelementary blends of this invention, a new methodology of using augments classical least squares (CLS) is applied. The use of CLS as an analytical method confirms and enhances the analysis of clear regions, cloudy regions or even birefringent regions, the latter two of which indicate areas of concern in a coating and are highly suggestive of a process or miscibility problem. Accordingly, stated simply, a phase-separated structure of immiscible blends can be directly observed with microscopy. A simple method used in the present invention to check the miscibility involves mixing the polymers and forming a thin film of about 10 micrometers to about 50 micrometers thick. If such a film is generally as clear and transparent as the least clear and transparent film of the same thickness of the individual polymers prior to blending, then the polymers are completely miscible.

Miscibility between polymers depends on many factors, including on the interactions between them and their molecular structures and molecular weights. The interaction between polymers can be characterized by the so-called Flory-Huggins parameter (χ). When .chi. is close to zero (0) or even is negative, the polymers are very likely miscible. Theoretically, χ can be estimated from the solubility parameters of the polymers, i.e., χ is proportional to the squared difference between them. Therefore, the miscibility of polymers can be approximately predicted. For example, the closer the solubility parameters of the two polymers are the higher the possibility that the two polymers are miscible. Miscibility between polymers tends to decrease as their molecular weights increases.

Thus in addition to the experimental determinations, the miscibility between polymers can be predicted simply based on the Flory-Huggins interaction parameters, or even more simply, based the solubility parameters of the components. However, because of the molecular weight effect, close solubility parameters do not necessarily guarantee miscibility.

It should be understood that a mixture of polymers needs only to meet one of the definitions provided herein to be miscible. Furthermore, a mixture of polymers may become a miscible blend upon incorporation of a bioactive agent and/or by the manner of the blend, i.e. a fine spray or an admixture. Certain embodiments of the present invention includes segmented polymers. As used herein, a “segmented polymer” is composed of multiple blocks, each of which can separate into the phase that is primarily composed of itself. As used herein, a “hard” segment or “hard” phase of a polymer is one that is either crystalline at use temperature or amorphous with a glass transition temperature above use temperature (i.e., glassy), and a “soft” segment or “soft” phase of a polymer is one that is amorphous with a glass transition temperature below use temperature (i.e., rubbery). Herein, a “segment” refers to the chemical formulation and “phase” refers to the morphology, which primarily includes the corresponding segment (e.g., hard segments form a hard phase), but can include some of the other segment (e.g., soft segments in a hard phase).

As used herein, a “hard” phase of a blend includes primarily a segmented polymer's hard segment and optionally at least part of a second polymer blended therein. Similarly, a “soft” phase of a blend includes predominantly a segmented polymer's soft segment and optionally at least part of a second polymer blended therein. Preferably, miscible blends of polymers of the present invention include blends of segmented polymers' soft segments.

When referring to the solubility parameter of a segmented polymer, “segment” is used and when referring to Tg of a segmented polymer, “phase” is used. Thus, the solubility parameter, which is typically a calculated value for segmented polymers, refers to the hard and/or soft segment of an individual polymer molecule, whereas the Tg, which is typically a measured value, refers to the hard and/or soft phase of the bulk polymer.

The types and amounts of polymers and active agents are typically selected to form a system having a preselected dissolution time through a preselected critical dimension of the miscible polymer blend. Glass transition temperatures, swellabilities, and solubility parameters of the polymers can be used in guiding one of skill in the art to select an appropriate combination of components in a coating composition, whether the bioactive agent is incorporated into the miscible polymer blend or not. Solubility parameters are generally useful for determining miscibility of the polymers and matching the solubility of the bioactive agent to that of the miscible polymer blend. Glass transition temperatures and/or swellabilities are generally useful for tuning the dissolution time (or rate) of the bioactive agent. These concepts are discussed in greater detail below.

A miscible polymer blend can be used in combination with an active agent in the delivery systems of the present invention in a variety of formats as long as the miscible polymer blend controls the delivery of the bioactive agent.

In one embodiment, a miscible polymer blend has one or more bioactive agents incorporated therein. Preferably, such an active agent is dissoluted predominantly under permeation control, which requires at least some solubility of the bioactive agents in the continuous portion (i.e., the miscible portion) of the polymer blend, whether the majority of the bioactive agent is loaded in the continuous portion or not. Dispersions are acceptable as long as little or no porosity channeling occurs during dissolution of the bioactive agents and the size of the dispersed domains is much smaller than the critical dimension of the blends, and the physical properties are generally uniform throughout the composition for desirable mechanical performance. This embodiment is often referred to as a “matrix” system.

In another embodiment, a miscible polymer blend initially provides a barrier to permeation of the one or more bioactive agents. This embodiment is often referred to as a “reservoir” system. A reservoir system can be in many formats with two or more layers. For example, a miscible polymer blend can form an outer layer over an inner layer of another material (referred to herein as the inner matrix material). In another example, a reservoir system can be in the form of a core-shell, wherein the miscible polymer blend forms the shell around the core matrix (i.e., the inner matrix material). At least initially upon formation, the miscible polymer blend in the shell or outer layer could be substantially free of bioactive agent. Subsequently, the one or more bioactive agents permeate from the inner matrix and through the miscible polymer blend for delivery to the subject. In one embodiment, the inner matrix material can be the active agent itself.

For a reservoir system, the release rate of the bioactive agent can be tuned with selection of the material of the outer layer. The inner matrix can include an immiscible mixture of polymers or it can be a homopolymer if the outer layer is a miscible blend of polymers.

As with matrix systems, the bioactive agent in a reservoir system is preferably dissoluted predominantly under permeation control through the miscible polymer blend of the barrier layer (i.e., the barrier polymer blend), which requires at least some solubility of the bioactive agent in the barrier polymer blend. Again, dispersions are acceptable as long as little or no porosity channeling occurs in the barrier polymer blend during dissolution of the bioactive agent and the size of the dispersed domains is much smaller than the critical dimension of the blends, and the physical properties are generally uniform throughout the barrier polymer blend for desirable mechanical performance.

In the coating compositions of the present invention, one or more bioactive agents are dissolutable through a miscible polymer blend. Dissolution is preferably controlled predominantly by permeation of the bioactive agents through the miscible polymer blend. That is, the bioactive agents initially dissolve into the miscible polymer blend and then diffuse through the miscible polymer blend predominantly under permeation control. Thus, as stated above, for certain preferred embodiments, the bioactive agents are at or below the solubility limit of the miscible polymer blend. Although not wishing to be bound by theory, it is believed that because of this mechanism the coating compositions of the present invention have a significant level of tunability.

If the one or more bioactive agent exceed the solubility of the miscible polymer blend and the amount of insoluble bioactive agents exceed the percolation limit, then the bioactive agents could be dissoluted predominantly through a porosity mechanism. In addition, if the largest dimension of the bioactive agents insoluble phase (e.g., particles or aggregates of particles) are on the same order as the critical dimension of the miscible polymer blend, then the bioactive agents could be dissoluted predominantly through a porosity mechanism. Dissolution by porosity control is typically undesirable because it does not provide effective predictability and controllability.

Because the coating compositions of the present invention preferably have a critical dimension on the micron-scale level, it can be difficult to include a sufficient amount of bioactive agent and avoid delivery by a porosity mechanism. Thus, the solubility parameters of the bioactive agent and at least one polymer of the miscible polymer blend are matched to maximize the level of loading while decreasing the tendency for delivery by a porosity mechanism.

One can determine if there is a permeation-controlled release mechanism by examining a dissolution profile of the amount of bioactive agent released versus time (t). For permeation-controlled release from a matrix system, the profile is directly proportional to t^{1/2}. For permeation-controlled release from a reservoir system, the profile is directly proportional to t. Alternatively, under sink conditions (i.e., conditions under which there are no rate-limiting barriers between the polymer blend and the media into which the active agent is dissoluted), porosity-controlled dissolution could result in a burst effect (i.e., an initial very rapid release of active agent).

The coating compositions of the present invention, whether in the form of a matrix system or a reservoir system, for example, without limitation, can be in the form of coatings on substrates (e.g., stents and catheters).

For preferred coating compositions of the present invention, the one or more bioactive agent are typically matched to the solubility of the miscible portion of the polymer blend. For embodiments of the invention in which the bioactive agents are hydrophobic, preferably at least one miscible polymer of the miscible polymer blend is hydrophobic. However, this is not necessarily required, and it may be undesirable to have a hydrophilic polymer in a coating composition for a low molecular weight hydrophilic active agent because of the potential for swelling of the polymers by water and the loss of controlled delivery of the bioactive agent. As used herein, in this context (in the context of the polymer of the blend), the term “hydrophobic” refers to a material that will not increase in volume by more than 10% or in weight by more than 10%, whichever comes first, when swollen by water at body temperature (i.e., about 37° C.).

As used herein, in this context (in the context of the bioactive agent), the term “hydrophilic” refers to a bioactive agent that has a solubility in water of more than 200 micrograms per milliliter. As used herein, in this context (in the context of the bioactive agent), the term “hydrophobic” refers to a bioactive agent that has a solubility in water of no more than 200 micrograms per milliliter.

As the size of the bioactive agent gets sufficiently large, diffusion through the polymer is affected. Thus, bioactive agents can be categorized based on molecular weights and polymers can be selected depending on the range of molecular weights of the active agents.

For certain preferred coating compositions of the present invention, the bioactive agent has a molecular weight of greater than about 1200 g/mol. For certain other preferred coating compositions of the present invention, the bioactive agent has a molecular weight of no greater than (i.e., less than or equal to) about 1200 g/mol. For even more preferred embodiments, bioactive agents of a molecular weight no greater than about 800 g/mol are desired.

Once the bioactive agent and the format for delivery (e.g., time/rate and critical dimension) are selected, one of skill in the art can utilize the teachings of the present invention to select the appropriate combination of at least two polymers to provide a coating composition.

As stated above, the types and amounts of polymers and active agents are typically selected to form a system having a preselected dissolution time (t) through a preselected critical dimension (x) of the miscible polymer blend. This involves selecting at least two polymers to provide a target diffusivity, which is directly proportional to the critical dimension squared divided by the time (x^{2/t}), for a given active agent.

In refining the selection of the polymers for the desired bioactive agent, the desired dissolution time (or rate), and the desired critical dimension, the parameters that can be considered when selecting the polymers for the desired bioactive agent include glass transition temperatures of the polymers, swellabilities of the polymers, solubility parameters of the polymers, and solubility parameters of the bioactive agents. These can be used in guiding one of skill in the art to select an appropriate combination of components in a coating composition, whether the bioactive agent is incorporated into the miscible polymer blend or not.

For enhancing the versatility of a permeation-controlled coating composition, for example, preferably the polymers are selected such that at least one of the following relationships is true: (1) the difference between the solubility parameter of the active agent and at least one solubility parameter of at least one polymer is no greater than about 10 J^{1/2}/cm^{3/2} (preferably, no greater than about 5 J^{1/2}/cm^{3/2}, and more preferably, no greater than about 3 J^{1/2}/cm^{3/2}); and (2) the difference between at least one solubility parameter of each of at least two polymers is no greater than about 5 J^{1/2}/cm^{3/2} (preferably, no greater than about 3 J^{1/2}/cm^{3/2}). More preferably, both relationships are true. Most preferably, both relationships are true for all polymers of the blend.

Typically, a compound has only one solubility parameter, although certain polymers, such as segmented copolymers and block copolymers, for example, can have more than one solubility parameter. Solubility parameters can be measured or they are calculated using an average of the values calculated using the Hoy Method and the Hoftyzer-van Krevelen Method (chemical group contribution methods), as disclosed in D. W. van Krevelen, Properties of Polymers, 3.sup.rd Edition, Elsevier, Amsterdam. To calculate these values, the volume of each chemical is needed, which can be calculated using the Fedors Method, disclosed in the same reference.

Solubility parameters can also be calculated with computer simulations, for example, molecular dynamics simulation and Monte Carlo simulation. Specifically, the molecular dynamics simulation can be conducted with Accelrys Materials Studio, Accelrys Inc., San Diego, Calif. The computer simulations can be used to directly calculate the Flory-Huggins parameter.

Examples of solubility parameters for various polymers and bioactive agents is shown in Table 1 below.

TABLE 1
Molecular weight, Tg and solubility parameters of PEVA/PAMA, sample drugs that
have been used in PEVA/PAMA blend technology and some solvents that dissolve PEVA and
PAMA (e.g. PBMA).

| | | | Solubility | Solubility parameter as | |
|---|---|---|---|---|---|
| | | Calculated | parameter as | referenced in D.W. van | |
| | Molecular | solubility | determined by | Krevelen. Properties of | |
| | weight | parameter | vaporization | Polymers, 3^{rd} ed. | |
| | (g/mole) | Joules^{1/2} cm^{3/2} | energy | Elsevier 1990. | Tg |
| PEVA | | | | 19.1/22.6 | ≈−30° C. |
| PBMA | | | | 17.8/18.4 | ≈24° C. |
| THF | | | 18.6 | | |
| Chloroform | | | 19.0 | | |
| Cyclohexane | | | 16.8 | | |
| Toluene | | | 18.2 | | |
| Rapamycin | 914.72 | 20.4 | | | |
| Triamcinolone | 434.5 | 22.8 | | | |
| acetonide | | | | | |
| estradiol | 272.4 | 21.5 | | | |
| dexamethasone | 392.5 | 23.7 | | | |
| cyclosporin | 1202.6 | 18.1 | | | |
| Taxol | 853.9 | 24.2 | | | |
| combretastatin | 316.3 | 22.1 | | | |

Source for Solubility Parameters:
1. D. W. van Krevelen, Properties of Polymers, 3rd ed., Elsevier, 1990. Table 7.5. Data were the average if there were two values listed in the sources.
2. Average of the calculated values based on Hoftyzer and van Kevelen's (H-vK) method (where the volumes of the chemicals were calculated based on Fedors' method) and Hoy's method. See Chapter 7, D. W. van Krevelen, Properties of Polymers, 3rd ed., Elsevier, 1990, for details of all the calculations, where Table 7.8 was for Hoftyzer and van Kevelen's method, Table 7.3 for Fedors' method, and Table 7.9 and 7.10 for Hoy's method.
   Source of Tg's (The Reported Value is the Average if There are Two Values Listed in the Sources):
1. Table 6.6, J. M. He, W. X. Chen, and X. X. Dong, Polymer Physics, revised version, FuDan University Press, ShangHai, China, 2000. Data were the average if there were two values listed in the sources.
2. Table 6.4, D. W. van Krevelen, Properties of Polymers, 3rd ed., Elsevier, 1990. Data were the average if there were two values listed in the sources.

In various embodiments of coating compositions in which the bioactive agent is hydrophobic, regardless of the molecular weight, polymers are typically selected such that the molar average solubility parameter of the miscible polymer blend is no greater than 28 J^{1/2}/cm^{3/2} (preferably, no greater than 25 J^{1/2}/cm^{3/2}). Herein “molar average solubility parameter” means the average of the solubility parameters of the blend components that are miscible with each other and that form the continuous portion of the miscible polymer blend. These are weighted by their molar percentage in the blend, without the bioactive agent incorporated into the polymer blend.

For example, for a hydrophobic bioactive agent of no greater than about 1200 g/mol, such as dexamethasone, which has a solubility parameter of 27 J^{1/2}/cm^{3/2}, based on Group Contribution Methods or 21 J^{1/2}/cm^{3/2} based on Molecular Dynamics Simulations, one polymer blend includes polyethylene-co-vinyl acetate (PEVA) and polybutylmethacrylate (PBMA). These have solubility parameters of 22.6 J^{1/2}/cm^{3/2} and 18.4 J^{1/2}/cm^{3/2}, respectively. A suitable blend of these polymers (1:1 molar ratio is PEVA/PBMA) has a molar average solubility parameter of 20.5 J.sup.1/2/cm.sup.3/2. This value was calculated as described herein as 22.6*0.5+18.5*0.5=20.5 (J^{1/2}/cm^{3/2}).

For delivery systems in which the bioactive agent is hydrophilic, regardless of the molecular weight, polymers are typically selected such that the molar average solubility parameter of the miscible polymer blend is greater than 21 J^{1/2}/cm^{3/2} (preferably, greater than 25 J^{1/2}/cm^{3/2}).

For enhancing the tunability of permeation-controlled dissolution times (rates) for low molecular weight active agents, preferably the polymers can be selected such that the difference between at least one Tg of at least two of the polymers corresponds to a range of diffusivities that includes the target diffusivity.

Alternatively, for enhancing the tunability of permeation-controlled dissolution times (rates) for high molecular weight active agents, preferably the polymers can be selected such that the difference between the swellabilities of at least two of the polymers of the blend corresponds to a range of diffusivities that includes the target diffusivity. The target diffusivity is determined by the preselected time (t) for delivery and the preselected critical dimension (x) of the polymer composition and is directly proportional to x^{2/t}.

The target diffusivity can be easily measured by dissolution analysis using the following equation (see, for example, Kinam Park edited, Controlled Drug Delivery: Challenges and Strategies, American Chemical Society, Washington, D.C., 1997): D=(Mₜ/4M_{∞})² (πx²/t) wherein D=diffusion coefficient; Mₜ=cumulative release; M_{∞}=total loading of active agent; x=the critical dimension (e.g., thickness of the film); and t=the dissolution time. This equation is valid during dissolution of up to 60 percent by weight of the initial load of the active agent. Also, blend samples should be in the form of a film.

Generally, at least one polymer has a bioactive agent diffusivity higher than the target diffusivity and at least one polymer has a bioactive agent diffusivity lower than the target diffusivity. The diffusivity of a polymer system can be easily measured by dissolution analysis. The diffusivity of a bioactive agent from each of the individual polymers can be determined by dissolution analysis, but can be estimated by relative Tg's or swellabilities of the major phase of each polymer.

The diffusivity can be correlated to glass transition temperatures of hydrophobic or hydrophilic polymers, which can be used to design a coating composition for low molecular weight bioactive agents (e.g., those having a molecular weight of no greater than about 1200 g/mol). Alternatively, the diffusivity can be correlated to swellabilities of hydrophobic or hydrophilic polymers, which can be used to design a coating composition for high molecular weight polymers (e.g., those having a molecular weight of greater than about 1200 g/mol). This is advantageous because the range of miscible blends can be used to encompass very different dissolution rates for bioactive agents of similar solubility.

The glass transition temperature of a polymer is a well-known parameter, which is typically a measured value. Exemplary values are listed in Table 1. For segmented polymers (e.g., a segmented polyurethane) the Tg refers to the particular phase of the bulk polymer. Typically, for low molecular weight bioactive agents, by selecting relatively low and high Tg polymers that are miscible, the dissolution kinetics of the system can be tuned. This is because a small molecular weight agent (e.g., no greater than about 1200 g/mol) diffuses through a path that is directly correlated with the Tg's, i.e., the free volume of the polymer blend is a linear function of the temperature with slope being greater when the temperature is above Tg.

Preferably, a polymer having at least one relatively high Tg is combined with a polymer having at least one relatively low Tg.

Swellabilities of polymers in water can be easily determined. It should be understood, however, that the swellability results from incorporation of water and not from an elevation in temperature. Typically, for high molecular weight bioactive agents, by selecting relatively low and high swell polymers that are miscible, the dissolution kinetics of the system can be tuned. Swellabilities of polymers are used to design these coating compositions because water that diffuses into the polymer blend tends to increase the free volume for bioactive agents of relatively high molecular weight (e.g., greater than about 1200 g/mol) to diffuse out of the polymeric blend.

In some embodiments, a polymer having a relatively high swellability is combined with a polymer having a relatively low swellability. By combining such high and low swell polymers, the coating composition can be tuned for the desired dissolution time of the bioactive agent.

For a first group of active agents that are hydrophobic and have a molecular weight of no greater than about 1200 g/mol, the polymers for the miscible polymer blend are selected such that: the average molar solubility parameter of the miscible polymers of the blend is no greater than 28 J^{1/2}/Cm^{3/2} (preferably, no greater than 25 J^{1/2}/cm^{3/2}); and the swellability of the blend is no greater than 10% by volume.

Examples of suitable combinations of polymer blends for use with one or more bioactive agents may include a first and second polymer as described below. Various embodiments of the present invention include the miscible polymer blend suitable for use with bioactive agents include the following: a blend of a polyalkyl methacrylate and a polyethylene-co-vinyl acetate.

A first polymer component of this invention provides an optimal combination of various structural/functional properties, including hydrophobicity, durability, bioactive agent release characteristics, biocompatability, molecular weight, and availability (and cost).

Examples of suitable first polymers include poly(alkyl)(meth)acrylates, and in particular, those with alkyl chain lengths from 2 to 8 carbons, and with molecular weights from 50 kilodaltons to 900 kilodaltons. An example of a particularly preferred first polymer is poly n-butylmethacrylate. Such polymers are available commercially, e.g., from Aldrich, with molecular weights ranging from about 200,000 daltons to about 320,000 daltons, and with varying inherent viscosity, solubility, and form (e.g., as crystals or powder).

A second polymer component of this invention provides an optimal combination of similar properties, and particularly when used in admixture with the first polymer component. Examples of suitable second polymers are available commercially and include poly(ethylene-co-vinyl acetate) having vinyl acetate concentrations of between about 10% and about 50%, in the form of beads, pellets, granules, etc. (commercially available are 12%, 14%, 18%, 25%, 33%). pEVA co-polymers with lower percent vinyl acetate become increasingly insoluble in typical solvents, whereas those with higher percent vinyl acetate become decreasingly durable.

A particularly preferred polymer mixture for use in this invention includes mixtures of poly(butylmethacrylate) (pBMA) and poly(ethylene-co-vinyl acetate) co-polymers (pEVA). This mixture of polymers has proven useful with absolute polymer concentrations (i.e., the total combined concentrations of both polymers in the coating composition), of between about 0.25 and about 70 percent (by weight). It has furthermore proven effective with individual polymer concentrations in the coating solution of between about 0.05 and about 70 weight percent. In one preferred embodiment the polymer mixture includes poly(n-butylmethacrylate) (pBMA) with a molecular weight of from 100 kilodaltons to 900 kilodaltons and a pEVA copolymer with a vinyl acetate content of from 24 to 36 weight percent. In a particularly preferred embodiment the polymer mixture includes poly(n-butylmethacrylate) with a molecular weight of from 200 kilodaltons to 400 kilodaltons and a pEVA copolymer with a vinyl acetate content of from 30 to 34 weight percent. The concentration of the bioactive agent or agents dissolved or suspended in the coating mixture can range from 0.01 to 90 percent, by weight, based on the weight of the final coating composition.

The bioactive (e.g., pharmaceutical) agents useful in the present invention include virtually any therapeutic substance which possesses desirable therapeutic characteristics for application to the implant site. These agents include: thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, peptides, proteins, enzymes, extracellular matrix components, ACE inhibitors, free radical scavengers, chelators, antioxidants, anti polymerases, antiviral agents, photodynamic therapy agents, and gene therapy agents.

A coating composition of this invention is preferably used to coat an implantable medical device that undergoes flexion or expansion in the course of its implantation or use in vivo. The words “flexion” and “expansion” as used herein with regard to implantable devices will refer to a device, or portion thereof, that is bent (e.g., by at least 45 degrees or more) and/or expanded (e.g., to more than twice its initial dimension), either in the course of its placement, or thereafter in the course of its use in vivo.

Examples of suitable catheters include urinary catheters, which would benefit from the incorporation of antimicrobial agents (e.g., antibiotics such as vancomycin or norfloxacin) into a surface coating, and intravenous catheters which would benefit from antimicrobial agents and or from antithrombotic agents (e.g., heparin, hirudin, coumadin). Such catheters are typically fabricated from such materials as silicone rubber, polyurethane, latex and polyvinylchloride.

The coating composition can also be used to coat stents, e.g., either self-expanding stents (such as the Wallstent variety), or balloon-expandable stents (as are available in a variety of styles, for instance, Gianturco-Roubin, Palmaz-Shatz, Wiktor, Strecker, ACS Multi-Link, Cordis, AVE Micro Stent), which are typically prepared from materials such as stainless steel or tantalum.

A coating composition of the present invention can be used to coat an implant surface using any suitable means, e.g., by dipping, spraying and the like. The suitability of the coating composition for use on a particular material, and in turn, the suitability of the coated composition can be evaluated by those skilled in the art, given the present description.

The overall weight of the coating upon the surface is typically not important. The weight of the coating attributable to the bioactive agent is preferably in the range of about 0.05 mg to about 10 mg of bioactive agent per cm² of the gross surface area of the device. More preferably, the weight of the coating attributable to the bioactive is between about 1 mg and about 5 mg of bioactive agent per cm² of the gross surface area of the device. This quantity of drug is generally required to provide adequate activity under physiological conditions.

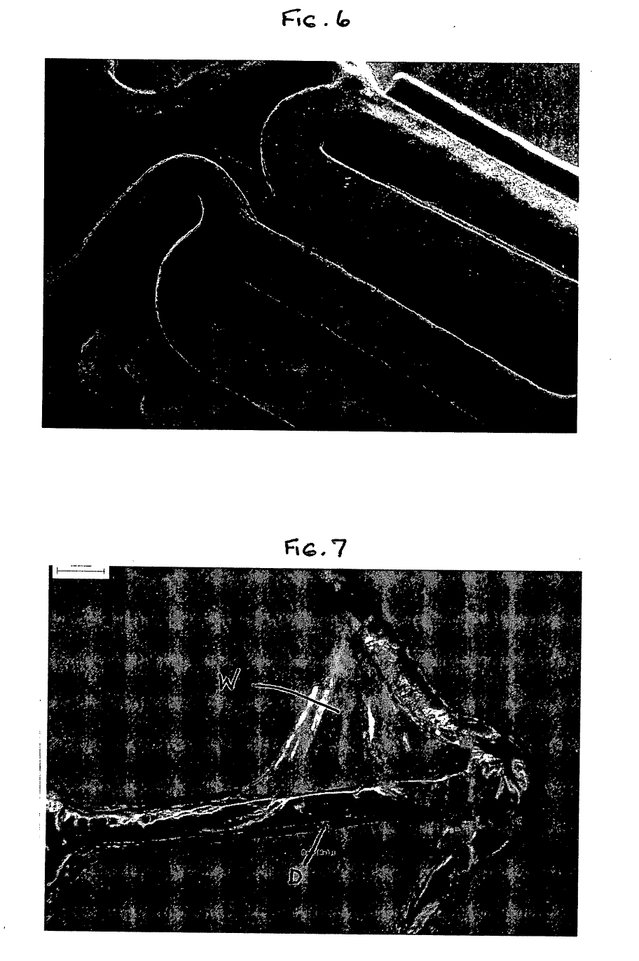

In turn, the outer diameter coating thickness of a presently preferred composition will typically be in the range of about 5 micrometers to about 100 micrometers. This level of coating thickness is generally required to provide an adequate density of drug to provide adequate activity under physiological conditions. The image of FIG. 6 shows one example of a smooth, substantially defect-free coating of the invention on a stent. FIG. 7, however, shows a stent having an outer diameter coating thickness of about 15-24 micrometers and comprising a polymer system not in accordance with the present invention. When the individual elements of the stent shown in FIG. 7 were subjected to flexion, the coating created a highly undesirable webbing (W) condition which is conducive, in that device, to the development of thrombosis. FIG. 7 also shows an area (D) of delamination of the polymer from the device substrate. This too leads to sub-optimal results for the user of the stent.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by the embodiments described by the language of the claims and the equivalents of those embodiments. Unless otherwise indicated, all percentages are by weight.

### EXAMPLES

### Test Methods

The potential suitability of particular coated compositions for in vivo use can be determined by a variety of methods, including the Durability, Flexibility and Release Tests, examples of each of which are described herein.

### Sample Preparation

One millimeter diameter stainless steel wires (e.g. 304 grade) are cut into 5 centimeter lengths. The wire segments can be Parylene treated or evaluated with no treatment. The wire segments are weighed on a micro-balance.

Bioactive agent/polymer mixtures are prepared at a range of concentrations in an appropriate solvent, in the manner described herein. The coating mixtures are applied to respective wires, or portions thereof, by dipping or spraying, and the coated wires are allowed to cure by solvent evaporation. The coated wires are re-weighed. From this weight, the mass of the coating is calculated, which in turn permits the mass of the coated polymer(s) and bioactive agent to be determined. The coating thickness can be measured using any suitable means, e.g., by the use of a microprocessor coating thickness gauge (Minitest 4100).

The Durability and Flexibility of the coated composition can be determined in the following manner.

### Durability Test

A suitable Durability Test, involves a method in which a coated specimen (e.g., wire) is subjected to repeated frictional forces intended to simulate the type of wear the sample would be exposed to in actual use, such as an implantable device undergoing flexion and/or expansion in the course of its implantation or use.

The Test described below employs a repetitive 60 cycle treatment, and is used to determine whether there is any change in force measurements between the first 5 cycles and the last 5 cycles, or whether there is any observable flaking or scarring detectable by scanning electron microscopy (“SEM”) analysis. Regenerated cellulose membrane is hydrated and wrapped around a 200 gram stainless steel sled. The cellulose membrane is clipped tightly on the opposite side of the sled. The sled with rotatable arm is then attached to a 250 gram digital force gauge with computer interface. The testing surface is mounted on a rail table with micro-stepper motor control. The wires are clamped onto the test surface. The cellulose covered sled is placed on top of the wires. Initial force measurements are taken as the sled moves at 0.5 cm/sec over a 5 cm section for 5 push/pull cycles. The sled then continues cycling over the coated samples for 50 push/pull cycles at 5 cm/sec to simulate abrasion. The velocity is then reduced to 0.5 cm/sec and the final force measurements are taken over another 5 push/pull cycles.

SEM micrographs are taken of abraded and nonabraded coated wires to evaluate the effects of the abrasion on the coating.

### Flexibility Test

A suitable Flexibility Test, in turn, can be used to detect imperfections (when examined by scanning electron microscopy) that develop in the course of flexing of a coated specimen, an in particular, signs of cracking at or near the area of a bend.

A wire specimen is obtained and coated in the manner described above. One end of the coated wire (1.0 cm) is clamped in a bench vice. The free end of the wire (1.0 cm) is held with a pliers. The wire is bent until the angle it forms with itself is less than 90 degrees. The wire is removed from the vice and examined by SEM to determine the effect of the bending on the coating.

### Bioactive Agent Release Assay

A suitable Bioactive Agent Release Assay, as described herein, can be used to determine the extent and rate of drug release under physiological conditions. In general it is desirable that less than 50% of the total quantity of the drug released, be released in the first 24 hours. It is frequently desirable for quantities of drug to be released for a duration of at least 30 days. After all the drug has been released, SEM evaluation should reveal a coherent and defect free coating.

Each coated wire is placed in a test tube with 5 mls of PBS. The tubes are placed on a rack in an environmental orbital shaker and agitated at 37° C. At timed intervals, the PBS is removed from the tube and replaced with fresh PBS. The drug concentration in each PBS sample is determined using the appropriate method.

After all measurable drug has been released from the coated wire, the wire is washed with water, dried, re-weighed, the coating thickness re-measured, and the coating quality examined by SEM analysis.

### Example 1

### Release of Hexachlorophene from Coated Stainless Steel Wires

A one millimeter diameter stainless steel wire (304 grade) was cut into two centimeter segments. The segments were treated with Parylene C coating composition (Parylene is a trademark of the Union Carbide Corporation). This treatment deposits a thin, conformal, polymeric coating on the wires.

Four solutions were prepared for use in coating the wires. The solutions included mixtures of: pEVA (33 weight percent vinyl acetate, from Aldrich Chemical Company, Inc.); poly(butyl methacrylate “pBMA”) (337,000 average molecular weight, from Aldrich Chemical Company, Inc.); and hexachlorophene (“HCP”) from Sigma Chemical Co., dissolved in tetrahydrofuran. The solutions were prepared as follows:
1) 10 mg/ml pEVA//60 mg/ml pBMA//100 mg/ml HCP
2) 35 mg/ml pEVA//35 mg/ml pBMA//100 mg/ml HCP
3) 60 mg/ml pEVA//10 mg/ml pBMA//100 mg/ml HCP
4) 0 mg/ml pEVA//0 mg/ml pBMA//100 mg/ml HCP

Nine wire segments were coated with each coating solution. The following protocol was followed for coating the wire segments. The Parylene-treated wire segments were wiped with an isopropyl alcohol dampened tissue prior to coating. The wire segments were dipped into the coating solution using a 2 cm/second dip speed. The wire segments were immediately withdrawn from the coating solution at a rate of 1 cm/second, after which the coated segments were air-dried at room temperature.

Individual wire segments were placed in tubes containing 2 ml of phosphate buffered saline (“PBS”, pH 7.4). The tubes were incubated at 37 degrees centigrade on an environmental, orbital shaker at 100 rotations/minute. The PBS was changed at 1 hour, 3 hours, and 5 hours on the first day, and daily thereafter. The PBS samples were analyzed for HCP concentration by measuring the absorbance of the samples at 298 nms on a UV/visible light spectrophotometer and comparing to an HCP standard curve.

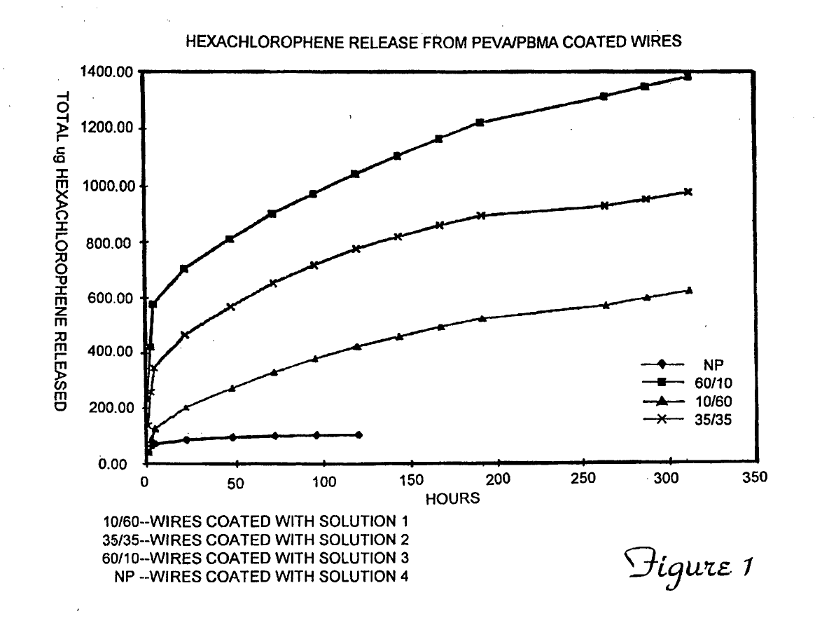

Results are provided in FIG. 1, which demonstrates the ability to control the elution rate of a pharmaceutical agent from a coated surface by varying the relative concentrations of a polymer mixture described by this invention.

### Example 2

The polymers described in this disclosure have been evaluated using an Assay protocol as outlined above. The polymer mixtures evaluated have ranged from 100% pBMA to 100% pEVA. Representative results of those evaluations are summarized below.

Control coatings that are made up entirely of pBMA are very durable showing no signs of wear in the Durability Test. When subjected to the Flexibility Test, however, these coatings develop cracks, particularly in the presence of significant concentrations of drug. These coatings also release drug very slowly.

Control coatings that are made up entirely of pEVA, in contrast, are less durable and show no signs of cracking in the Flexibility Test, but develop significant scarring in the Durability Test. These coatings release drugs relatively rapidly, usually releasing more than 50% of the total within 24 hours.

Coatings of the present invention, which contain a mixture of both polymers, are very durable, with no signs of wear in the Durability Test and no cracking in the Flexibility Test. Drug release from these coatings can be manipulated by varying the relative concentrations of the polymers. For instance, the rate of drug release can be controllably increased by increasing the relative concentration of pEVA.

Bioactive agent containing coatings which show no signs of scarring in the Durability Test and no cracking in the Flexibility Test possess the characteristics necessary for application to implantable medical devices that undergo flexion and/or expansion in the course of implantation and/or use.

1. A coating having a target diffusivity, the system comprising a bioactive agent and a miscible polymer blend; wherein: the bioactive agent is hydrophobic and has a molecular weight of no greater than about 1200 g/mol; and the miscible polymer blend comprises at least two polymers, each with at least one solubility parameter, wherein: the difference between the solubility parameter of the bioactive agent and at least one solubility parameter of at least one of the polymers is no greater than about 10 J^{1/2}/cm^{3/2}, and the difference between at least one solubility parameter of each of at least two polymers is no greater than about 5 J^{1/2}/cm^{3/2}; at least one polymer has an active agent diffusivity higher than the target diffusivity and at least one polymer has a bioactive agent diffusivity lower than the target diffusivity; the molar average solubility parameter of the blend is no greater than 25 J^{1/2}/cm^{3/2}; and the swellability of the blend is no greater than 10% by volume.

2. The coating of claim 1 wherein: the miscible polymer blend includes a blend of a polyalkyl methacrylate and a polyethylene-co-vinyl acetate.

3. The coating of claim 1 wherein the difference between at least one Tg of at least two of the polymers corresponds to a range of diffusivities that includes the target diffusivity.

4. The coating of claim 1 wherein the bioactive agent is incorporated within the miscible polymer blend.

5. The coating of claim 1 wherein the miscible polymer blend initially provides a barrier for permeation of the bioactive agent.

6. The coating of claim 6 wherein the active agent is incorporated within an inner matrix.

7. The coating of claim 1 wherein the miscible polymer blend includes at least two hydrophobic polymers.

8. The coating of claim 1 wherein the difference between the solubility parameter of the bioactive agent and at least one solubility parameter of at least one of the polymers is no greater than about 5 J^{1/2}/cm^{3/2}.

9. The coating of claim 1 wherein the difference between at least one solubility parameter of each of at least two of the polymers is no greater than about 3 J^{1/2}/cm^{3/2}.

10. A coating having a target diffusivity, the system comprising a bioactive agent and a miscible polymer blend; wherein: the bioactive agent is hydrophilic and has a molecular weight of no greater than about 1200 g/mol; and the miscible polymer blend comprises at least two polymers, wherein: the difference between the solubility parameter of the bioactive agent and at least one solubility parameter of at least one of the polymers is no greater than about 10 J^{1/2}/cm^{3/2}, and the difference between at least one solubility parameter of each of at least two polymers is no greater than about 5 J^{1/2}/cm^{3/2}; at least one polymer has a bioactive agent diffusivity higher than the target diffusivity and at least one polymer has a bioactive agent diffusivity lower than the target diffusivity; the molar average solubility parameter of the blend is greater than 25 J^{1/2}/cm^{3/2}; and the swellability of the blend is no greater than 10% by volume.

11. The coating of claim 10 wherein the miscible polymer blend includes a blend of a polyalkyl methacrylate and a polyethylene-co-vinyl acetate.

12. The coating of claim 10 wherein the difference between at least one Tg of at least two of the polymers corresponds to a range of diffusivities that includes the target diffusivity.

13. The coating of claim 10 wherein the bioactive agent is incorporated within the miscible polymer blend.

14. The coating of claim 10 wherein the miscible polymer blend initially provides a barrier for permeation of the bioactive agent.

15. The coating of claim 14 wherein the bioactive agent is incorporated within an inner matrix.

16. The coating of claim 10 wherein the miscible polymer blend includes at least two hydrophobic polymers.

17. The coating of claim 10 wherein the difference between the solubility parameter of the bioactive agent and at least one solubility parameter of at least one of the polymers is no greater than about 5 J^{1/2}/cm^{3/2}.

18. The coating of claim 10 wherein the difference between at least one solubility parameter of each of at least two of the polymers is no greater than about 3 J^{1/2}/cm^{3/2}.

19. A medical device comprising the coating of claim 1.

20. The medical device of claim 19 selected from the group consisting of a stent and catheter.

21. A medical device comprising the coating of claim 10.

22. The medical device of claim 21 selected from the group consisting of a stent and catheter.

23. A method of designing a coating for delivering an active agent over a preselected dissolution time (t) through a preselected critical dimension (x) of a miscible polymer blend, the method comprising: providing a bioactive agent having a molecular weight no greater than about 1200 g/mol; selecting at least two polymers, wherein: the difference between the solubility parameter of the bioactive agent and at least one solubility parameter of each of the polymers is no greater than about 10 J^{1/2}/cm^{3/2}, and the difference between at least one solubility parameter of each of the at least two polymers is no greater than about 5 J^{1/2}/cm^{3/2}; and the difference between at least one Tg of each of the at least two polymers is sufficient to include the target diffusivity; combining the at least two polymers to form a miscible polymer blend; and combining the miscible polymer blend with the bioactive agent to form a coating having the preselected dissolution time through a preselected critical dimension of the miscible polymer blend.

24. The method of claim 23 wherein the bioactive agent is incorporated within the miscible polymer blend.

25. The method of claim 23 wherein miscible polymer blend initially provides a barrier for permeation of the bioactive agent.

26. The method of claim 23 wherein the bioactive agent is incorporated within an inner matrix.

27. The method of claim 23 wherein the bioactive agent is hydrophobic.

28. The method of claim 23 wherein the active agent is hydrophilic.

29. The method of claim 48 wherein: the miscible polymer blend includes a blend of a polyalkyl methacrylate and a polyethylene-co-vinyl acetate.

30. A method of designing a coating for delivering a bioactive agent over a preselected dissolution time (t) through a preselected critical dimension (x) of a miscible polymer blend, the method comprising: providing a bioactive agent having a molecular weight greater than about 1200 g/mol; selecting at least two polymers, wherein: the difference between the solubility parameter of the bioactive agent and at least one solubility parameter of each of the polymers is no greater than about 10 J^{1/2}/cm^{3/2}, and the difference between at least one solubility parameter of each of the at least two polymers is no greater than about 5 J^{1/2}/Cm^{3/2}; and the difference between the swellabilities of the at least two polymers is sufficient to include the target diffusivity; combining the at least two polymers to form a miscible polymer blend; and combining the miscible polymer blend with the bioactive agent to form a coating having the preselected dissolution time through a preselected critical dimension of the miscible polymer blend.

31. The method of claim 30 wherein the bioactive agent is incorporated within the miscible polymer blend.

32. The method of claim 30 wherein miscible polymer blend initially provides a barrier for permeation of the bioactive agent.

33. The method of claim 30 wherein the bioactive agent is incorporated within an inner matrix.

34. The method of claim 30 wherein the bioactive agent is hydrophobic.

35. The method of claim 30 wherein the active agent is hydrophilic.

36. The method of claim 30 wherein: the miscible polymer includes a blend of a polyalkyl methacrylate and a polyethylene-co-vinyl acetate.

37. A method for delivering a bioactive agent to a subject, the method comprising: providing the coating of claim 1; and administering the coating in a subject.

38. A method for delivering a bioactive agent to a subject, the method comprising: providing the coating of claim 10; and administering the coating in a subject.
